(19) United States
(12) Patent Application Publication
(10) Pub. No.: US 2022/0414416 A1
(43) Pub. Date: Dec. 29, 2022

(54) Real-Time Position Tracking and Alerting

(71) Applicant: Machine Mavericks, Kingston (CA)

(72) Inventors: Olivia Helena O'Driscoll, Kingston (CA); Ethan Glassford, Kingston (CA); Niall O'Driscoll, Kingston (CA)

(21) Appl. No.: 17/848,671

(22) Filed: Jun. 24, 2022

Related U.S. Application Data
(60) Provisional application No. 63/215,032, filed on Jun. 25, 2021.

(51) Int. Cl. G06K 19/077; G06F 3/01; H04W 4/029

(52) U.S. Cl. CPC G06K 19/07762; G06F 3/016; H04W 4/029

(57) ABSTRACT

A wearable device is described that is configured to alert a subject prior to encountering an obstacle. The subject may be visually-impaired. The device includes: a mobile transceiver tag adapted to be worn by a subject within a designated area (e.g., a swimming pool); three or more stationary transceiver beacons in spaced apart, fixed positions; a wearable signal output device (e.g., headset); and a processor with software that can calculate the subject's position relative to one or more obstacles (e.g., end of swimming lane) and sends a left, right, or reverse signal to the signal output device.

### RELATED APPLICATION

This application claims the benefit of the filing date of U.S. Application No. 63/215,032, filed on Jun. 25, 2021, the contents of which are incorporated herein by reference in their entirety.

### FIELD

The invention pertains to wearable devices that track the position of a subject in motion and alert the subject when an obstacle is being approached.

### BACKGROUND

Swimmers who swim lengths in a pool must be careful not to hit the wall at the pool's end. Injuries take place in child swimmers, visually-impaired swimmers and backstroke swimmers because of the difficulty of knowing when the wall is drawing near.

A coach or bystander is often employed to tap a visually-impaired swimmer, but that requirement for one or two extra people limits the independence of swimmers.

Technologies such as the Blind Cap by Samsung continue to require input from a bystander or swim coach to initiate a signal to the swimmer. In one technology that does not require the help of another person, a downward facing camera is worn strapped to a swimmer's thigh, which may be uncomfortable. The camera tracks the swimmer's position in a pool provided that there are easily legible lane markers (Muehlbradt, A. Proc. Of Assets'17 (2017) p. 377-378, DOI: http://dx.doi.org/10.1145/3132525.3134822).

There exists a need for a comfortable and reliable tracking and alert system for all swimmers to prevent injury.

### SUMMARY

In one aspect, the invention provides a device for alerting a subject in motion of an obstacle, comprising a mobile transceiver (“tag”) adapted to be worn by a subject within a designated area, a plurality of stationary transceivers (“beacons”) located in selected positions, wherein the beacons are spaced apart from each other above or around the designated area, and wherein the beacons are able to communicate with each other, a wearable signal output device, and a processor coupled to at least one non-transitory computer readable medium containing processing instructions that when executed by the processor cause the processor to receive data regarding the trilaterational position of the tag relative to at least three of the beacons, calculate in real-time the tag's position relative to one or more selected obstacle(s) in the designated area, and output a signal to the signal output device when the tag is approaching an undesirable location. In one embodiment, the subject is a swimmer and the undesirable location comprises a location of an obstacle. In one embodiment, the tag and plurality of beacons emit ultra-wideband or ultrasonic signals. In one embodiment, the subject is visually-impaired. In one embodiment, the signal output device is adapted to produce an audible signal. In one embodiment, the signal output device is adapted to produce a haptic signal. In one embodiment, the signal output device is located on or near the subject's temples or ears. In one embodiment, the output device is a bone conduction headset, bone conduction BLUETOOTH® headset, or a haptic signalling device. In one embodiment, the device further comprises an Inertial Measurement Unit (IMU). In one embodiment, the IMU is Baizuu L3GD20 LSM303D. In another embodiment, the IMU is a ten degree of freedom IMU by ST Micro.

In one aspect, the invention provides a system that includes a mobile transceiver (“tag”) adapted to be attached to a subject in a designated area, a plurality of stationary transceivers (“beacons”) that are in communication with the tag and with each other and are in spaced apart, fixed positions around the perimeter of the designated area, a signal output device configured to be worn by the subject, and a processor and a set of computer-readable instructions on a non-transitory computer-readable medium coupled to or associated with the processor that, when executed on the processor, cause the processor to receive data from the tag, calculate when the tag has passed a defined point and communicate with the signal output device to emit a signal. In one embodiment, the subject is a swimmer and the undesirable location comprises a location of an obstacle. In one embodiment, the tag and the plurality of beacons are adapted to emit and receive ultra-wideband or ultrasonic signals. In one embodiment, the subject is visually-impaired. In one embodiment, the output device is adapted to produce an audible signal. In one embodiment, the output device is adapted to produce a haptic signal. In one embodiment, the output device is located on or near the subject's temples or ears. In one embodiment, the output device is a bone conduction headset, bone conduction BLUETOOTH® headset, or a haptic signalling device. In one embodiment, the system further comprises an Inertial Measurement Unit (IMU).

In one aspect, the invention provides a method of tracking a subject in motion and alerting the subject, comprising disposing a mobile transceiver (“tag”) and a signal output device on a subject, disposing a plurality of stationary transceivers (“beacons”) in spaced apart, fixed positions around the perimeter of a designated area whereby the beacons and the tag are in communication substantially consistently, whereby tag location data is provided by trilateration, communicating the tag location data to a processor having a set of computer-readable instructions on a non-transitory computer- readable medium coupled to or associated with the processor that, when executed on the processor, direct the processor to compare the tag location data to stored parameters for an activity area, and sending one of a plurality of output signals to a signal output device if the tag is approaching a selected location. In one embodiment, the plurality of output signals comprises turn left, turn right, or reverse direction. In one embodiment, the tag and the plurality of beacons emit and receive ultra-wideband or ultrasonic signals.

In one aspect, the invention provides a computer executable programmed instructions stored on a non-transitory computer readable storage medium, wherein the programmed instructions direct a processor to receive data regarding the trilaterational position of a mobile transceiver (“tag”) relative to at least three of stationary transceivers (“beacons”) in a designated area, calculate the tag's position relative to stored parameters for one or more selected locations in the designated area, and output a signal to the signal output device when the tag is approaching a selected location.

### BRIEF DESCRIPTION OF THE DRAWINGS

For a better understanding of the invention and to show more clearly how it may be carried into effect, reference will now be made, by way of example, to the accompanying drawings, wherein:

FIGS. 1A and 1B show schematics indicating the location of tag 1, IMU 4, processor 5, and signal output device 6, relative to a swimmer's head wearing swim goggles according to one embodiment.

FIG. 2 shows a schematic indicating the location of tag 1, IMU 4, processor 5, and signal output device 6, relative to a swimmer's head wearing swim goggles and a swim cap according to one embodiment.

FIG. 3 shows a diagram indicating the location of four fixed stationary beacons 2 spaced out around the walls of a 25 m swimming pool, and five swimmers wearing tags 1 according to one embodiment.

FIG. 4 shows a diagram indicating the location of four stationary beacons 2 that are spaced out around the walls surrounding a swimming pool, a remote processor 3, and a swimmer wearing a tag 1, IMU 4, wearable processor 5, and signal output device 6 according to one embodiment.

FIG. 5 shows a diagram indicating the location of four stationary beacons 2 that are spaced out around the walls surrounding a swimming pool, a remote processor 3, and a swimmer wearing a tag 1, wearable processor 5, and signal output device 6 according to one embodiment.

FIG. 6 shows a diagram indicating the location of four stationary beacons 2 that are spaced out around the walls surrounding a swimming pool, and a swimmer wearing a tag 1, wearable processor 5, and signal output device 6 according to one embodiment.

FIG. 7A shows a flowsheet for an algorithm used in the computer program shown in FIG. 8 according to one embodiment.

FIG. 7B shows a flowsheet for an algorithm used in the computer program shown in FIG. 8 according to one embodiment.

FIG. 8 shows code of a computer program that collects the pool frame of reference, a swimming lane's frame of reference, stationary beacons' signals, and a tag's signals according to one embodiment.

### DETAILED DESCRIPTION OF EMBODIMENTS

A real-time location tracking and alert system is described herein. The system is useful for tracking a subject's location within a designated area and to provide an alert when the subject is approaching an obstacle.

The system includes a mobile transceiver that communicates through ultra-wideband (UWB or ultrasonic), which is referred to herein as a tag, which is worn on the subject's body. In one embodiment, the tag is worn on the subject's head. In some embodiments, ultra-wideband (UWB) transceivers were used to provide short-range, wireless communication via radio waves across a wide radio bandwidth (e,g., from 500 MHz to several gigahertz). In this way, UWB uses low power to enable locking onto an object and communicating data regarding its precise location. In other embodiments, ultrasonic transceivers were used to provide short-range, wireless communication via sound waves at a frequency above the range of human hearing.

A plurality of fixed stationary transceivers (UWB or ultrasonic), which are referred to herein as beacons, are located in selected positions around the perimeter of the activity area at a height or heights that enable at least three beacons to be in communication with the tag. If the tag is temporarily immersed in water, it will be out of communication with the beacons; communication will resume when the tag is above water. This communication by three beacons enables trilateration to be used to pinpoint the location of the tag. Trilateration is a method for calculating a position that relies on a known distance between three beacons and the measured distance from those three beacons to the tag (e.g., 2-way ranging). A corresponding receiver uses trilateration to pinpoint the tag's location within the activity area. In one embodiment, the receiver is located in the tag; and the tag sends the tag's location data to a processor. The processor may be remote from the subject, or it may be adapted to be wearable by the subject.

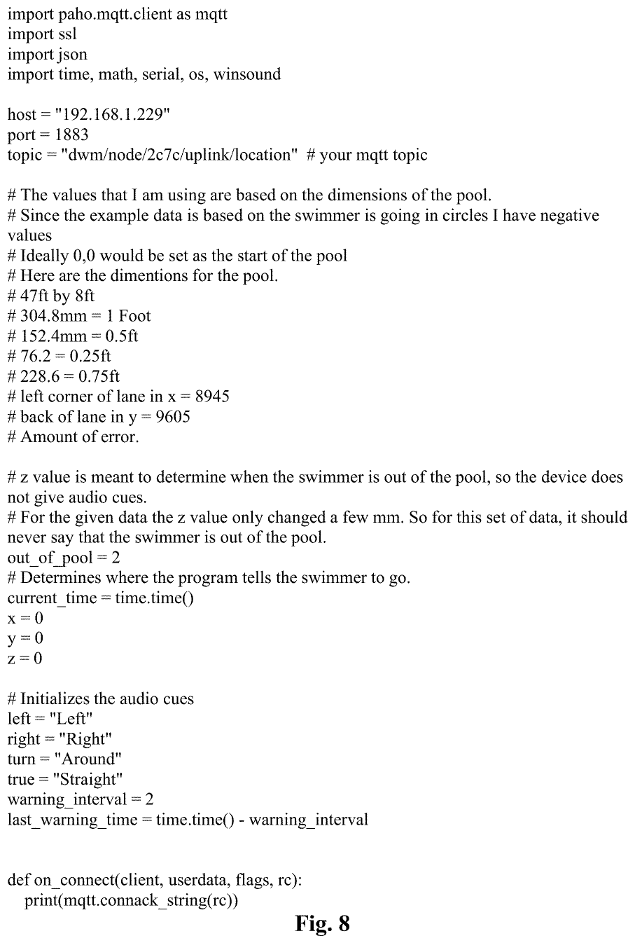

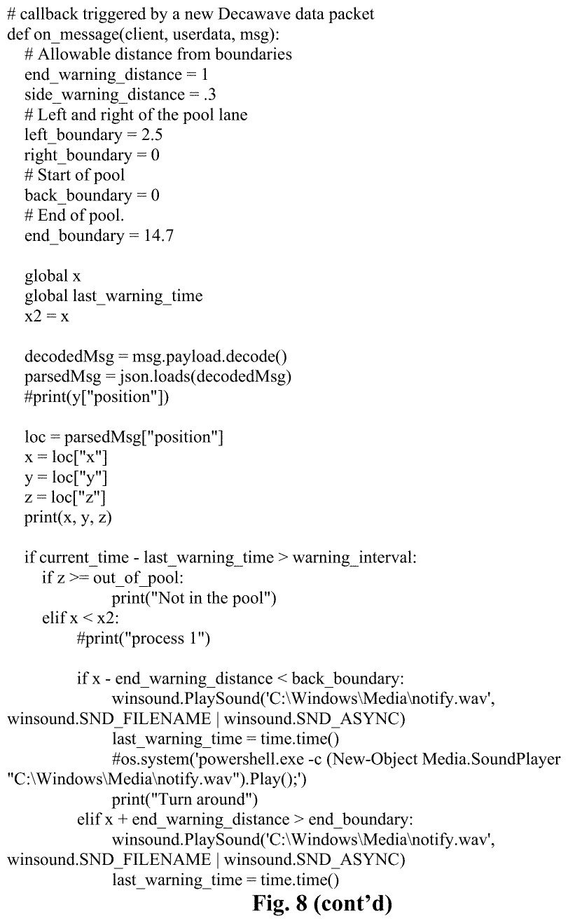

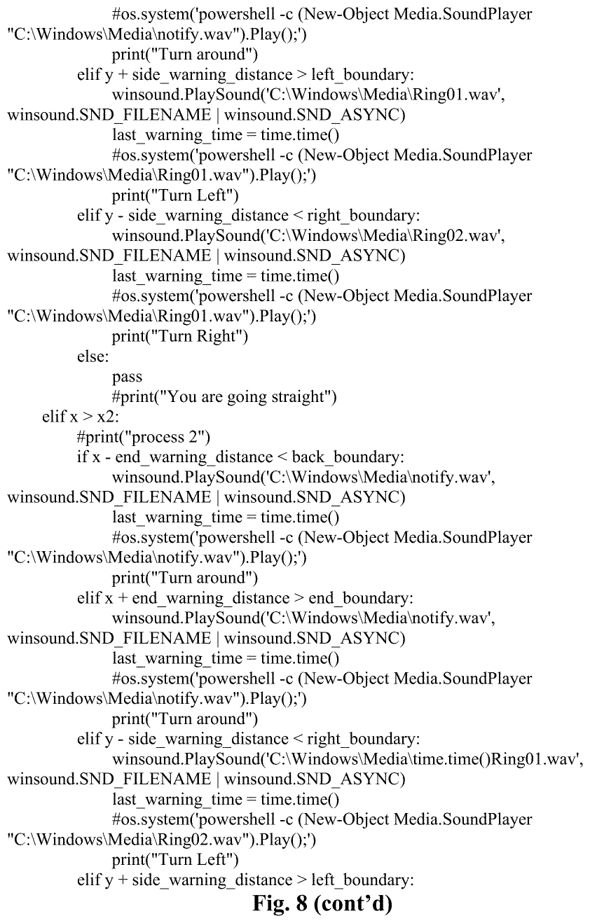

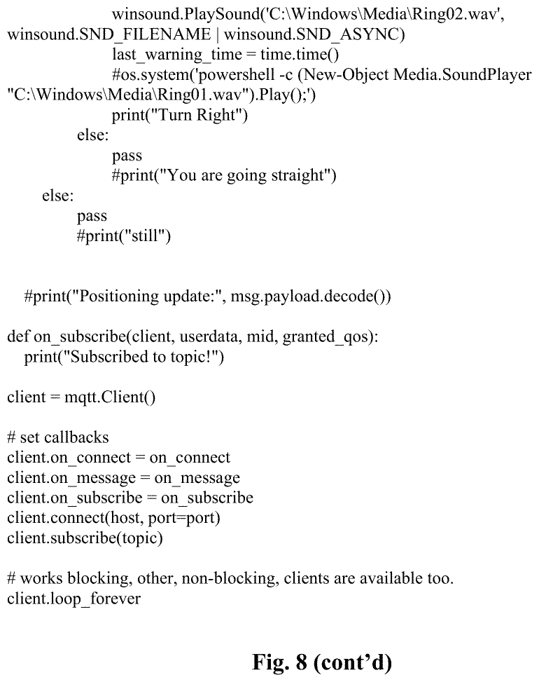

An external processor then receives data that results from the trilateration and compares location of the tag to previously entered positional data regarding obstacles within the activity area. A computer program (see FIG. 8) was created and implemented to determine whether the relative location of the tag to the obstacles merited an alert being sent to warn the subject. The signal to emit an auditory alert was received by a signal output device, which was worn by the subject. The auditory alert would be heard by the subject who could then change course accordingly.

In one embodiment, the system is configured to track the location of a subject who is a swimmer located in an activity area that is a swimming pool; and to provide the swimmer with an alert when part of the swimmer's body is approaching an obstacle (e.g., pool wall, bulkhead, danger zone, lane divider, other swimmers). The swimmer can then change course, which may involve a left or right heading or a reversal of direction if the obstacle is the wall at the end of the pool.

In one embodiment, the location tracking and alert system includes the following components: a tag 1; a plurality of beacons 2; an external processor 3; and a signal output device 6.

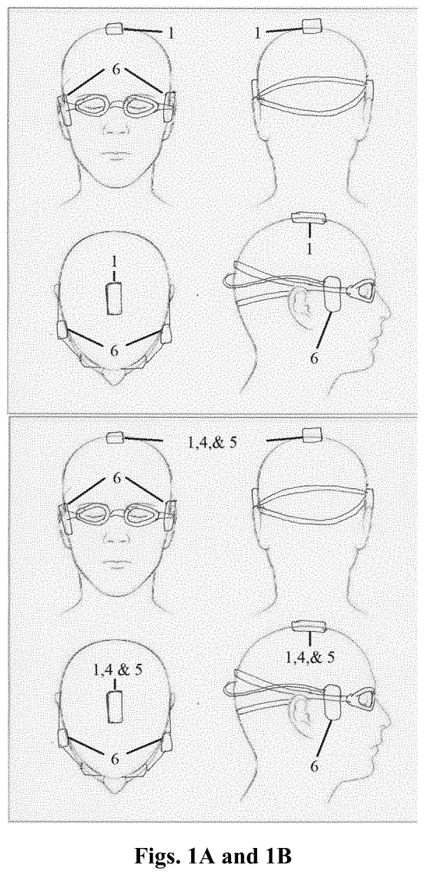

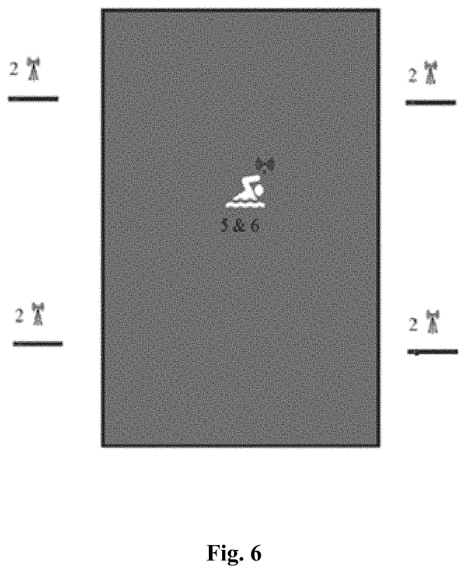

In another embodiment, the location tracking and alert system includes: a tag 1 worn by a subject in an activity area; a plurality of beacons 2 located around the perimeter of the activity area in an evenly spaced configuration; a remote processor 3; a wearable processor 5; and a signal output device 6 (see FIGS. 1 to 6). Examples of 5 include certain models of Apple Watch and Smartphones, which include a UWB or BLUETOOTH® transmitter. The inventors envision a wearable processor 5 that is adapted to communicate with 1, 2, and 6. Certain wearable processors include a UWB transceiver; the inventors envision an embodiment as shown in FIG. 6, wherein the tag 1 is located inside of 5. Notably, a Smartphone includes an inertial measurement unit (IMU) 4 through features and sensors such as, for example, an accelerometer and a gyroscope. An example of an IMU is Baizuu L3GD20 LSM303D. The IMU 4 can be used to augment the information about the location of the tag, in situations where the data regarding the tag's location has gaps. Such gaps may result if the tag is immersed in water during the activity of the tagged subject (e.g, the subject is swimming).

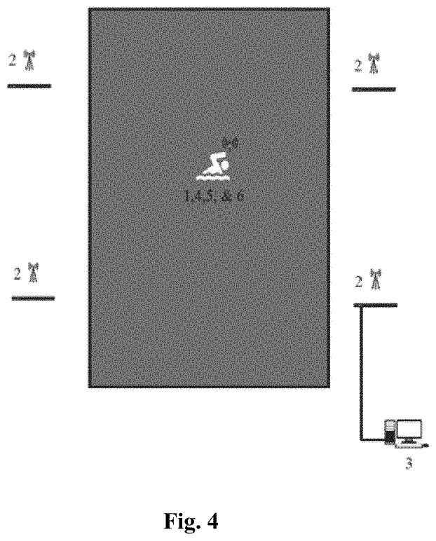

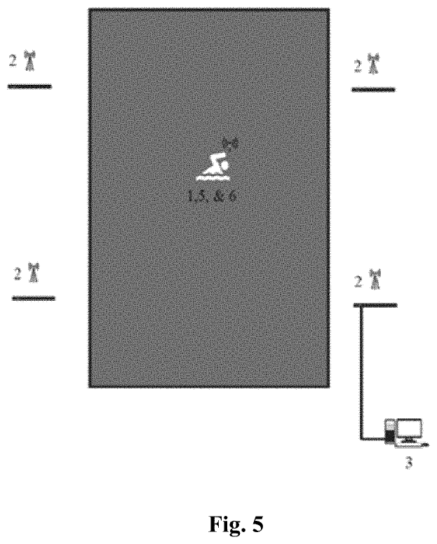

In operation, tag 1 is worn on the subject's body (see FIGS. 1A and 1B). A plurality of stationary beacons 2 are spaced out from one another in a fixed configuration around the perimeter of the activity area (see FIGS. 3 to 6. The tag 1 generates UWB pulses, and its location is pinpointed via trilateration using at least three of the stationary beacons 2. In one embodiment, 3 is located within or near the activity area (see FIGS. 4 and 5). Remote processor 3 receives positional data from the trilateration and calculates the relative position of the tag 1 to obstacles whose positions are pre-programmed. In this way, when the tag 1 is approaching an obstacle, a signal is sent to signal output device 6 causing it to emit a signal which alerts the subject. The subject then corrects course (e.g., turns left, right, reverses direction). In one embodiment, it is possible to vary the signal to indicate whether the subject should turn left, or turn right, or reverse direction.

In one embodiment, the system includes an inertial measurement unit (IMU) 4 that tracks a subject's motion (e.g., acceleration, rotation, etc.)(see FIG. 4). The IMU data can be used on its own to estimate position changes from one reading to another, or in combination with data obtained from the beacons 2 to give a more accurate position estimate than either the beacons 2 or the IMU 4 could give on their own.

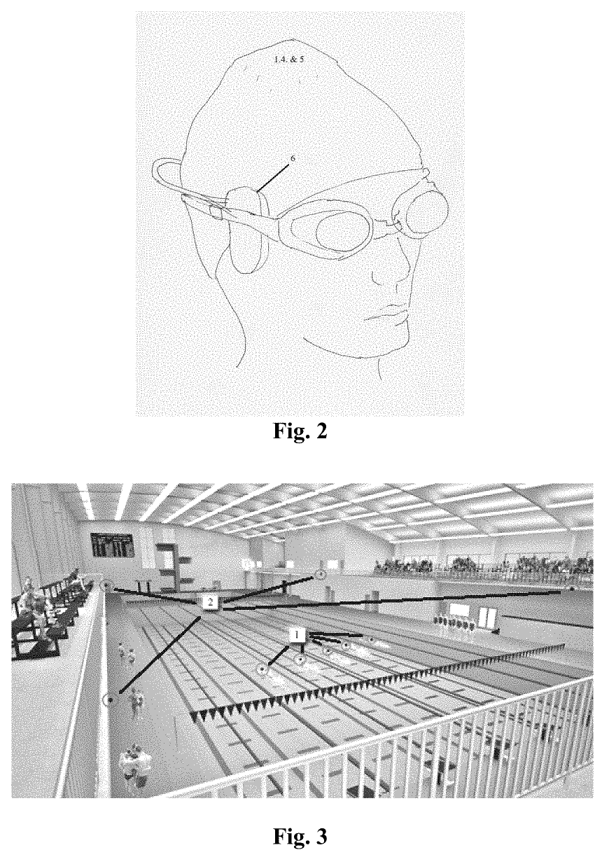

In one embodiment, the system includes a wearable processor 5 (e.g., Smartphone, APPLE WATCH®) that processes output from tag 1 and IMU 4. In one embodiment, tag 1, IMU 4, and wearable processor 5 are integrated into a single unit (see FIG. 4). This single unit may be worn attached to or under a swim cap (see FIGS. 1A, 1B, and 2). In one embodiment, communication between 1, 4, and 5 is wired. In one embodiment, communication between 1, 4, and 5 is wireless (e.g., BLUETOOTH®).

Also included in the system is a signal output device 6 (e.g., bone conduction headset, bone conduction BLUETOOTH® headset, haptic or vibrotactile signalling device, audio signalling device) that communicates processed information to the subject. In one embodiment, a wraparound strap of the signal output device 6, which is a feature of most bone conduction headsets, allows it to be worn in a more secure manner even during a swim.

In the embodiment wherein the system operates in a swimming pool environment, components 1, 4, 5 and 6 are encased in a waterproof casing and stationary beacons 2 are adapted to withstand a humid environment.

During setup, remote processor 3 identifies and saves X, Y, and Z coordinates of the locations of stationary beacons 2. The location and dimensions of the activity area (e.g., swimming pool) relative to these stationary beacons 2 are then entered. With this information entered, the location of tag 1 is triangulated relative to the stationary beacons 2, and is determined relative to the activity area. Then, if merited, an instruction is sent to signal output device 6 telling it to emit a signal for the subject to hear or feel.

In addition to the basic functionality of guiding a subject during an activity, the system can also gather and communicate additional information from other sensors. For example, by establishing a BLUETOOTH® connection to sensors such as a power meter, heart rate monitor, and/or a Global Positioning System (GPS), data from such sensors can be converted to audio and communicated to the subject via signal output device 6.

In an embodiment where the activity area is a swimming pool and the subject is a swimmer, coordinates of the swimming pool are set so that the starting corner of lane 1, at the water's surface, has an x, y, z location of (0,0,0) with the x axis running the length of the swimming pool, the y axis running the width of the swimming pool, and the z axis running the depth of the swimming pool. In operation, a swimmer who is wearing a tag 1 moves through the swimming pool. Information from 1 and stationary beacons 2 is sent to remote processor 3 and the x, y, z position of tag 1 is continuously calculated.

In certain embodiments, a wearable processor 5, which is in communication with tag 1, tracks the swimmer's progress through the pool. The swimmer's position may optionally be refined using IMU 4 using filtering techniques (e.g., Kalman filter, low-pass filter, moving average, etc.) to filter out high frequency noise in the position as a function of time. When the processor detects and/or calculates that the swimmer is approaching the end of the pool, it sends a signal to alert the swimmer via a signal output device 6. The alerted swimmer is then able to turn and avoid a collision. While the swimmer moves from one end of the pool to the other, the system can also track the swimmer's side to side position in the lane and similarly alert the swimmer to swim left or right in order to stay in the center of the swim lane. These signals could be customized depending on the individual swimmer's needs.

As a safety feature, the system is additionally able to provide an audible “heartbeat” signal to the swimmer indicating that the system is working correctly. If communication between the components fails and the data becomes unreliable, the heartbeat will stop thereby alerting the swimmer not to rely on the system until communication is restored.

In one embodiment, the system described herein identifies when the swimmer is in the pool and only gives output signals during that time.

The following working examples further illustrate the invention and are not intended to be limiting in any respect.

### Example 1A. UWB tracking and alerting a subject who is swimming lengths of a pool

One embodiment of the track and alert system that is detailed below. Four stationary beacons 2 were used, specifically, they were DWM1001 Ultra-wideband (UWB) beacons, which are available from QORVO (Greensboro, NC, USA). The beacons were powered by rechargeable batteries. An alternate supplier choice would be Creator Kit Lite's Developer Tag DW1000 beacon by Pozyx UWB (Ghent, Belgium).

Each beacon was mounted on posts and the posts were positioned so that two beacons were on one longitudinal side of the pool, and the other two posts were on the other longitudinal side of the pool (see FIG. 3). On each side, the posts were separated from one another by about ¾ of the pool length. Positioning the posts at the ends of the pool's swim lengths should be avoided to optimize accuracy of the swimmer's position relative to the ends of the pool.

A DWM1001 UWB tag 1 was housed in a waterproof container (i.e., a sealed ZIPLOC® bag) and inserted into a swim cap that was worn by the subject (see FIGS. 1 and 2). The mobile beacon was powered by a lithium polymer battery. The swimmer also wore a signal output device that was waterproof (i.e., BLUETOOTH®-enabled Aeropex bone conduction headset, which is available from AfterShokz, Austin, TX, USA). The headset was worn near the temples of the swimmer (see FIG. 2). The swimmer's ears were not covered by the signal output device to enable the swimmer to hear other swimmers, lifeguards, etc.

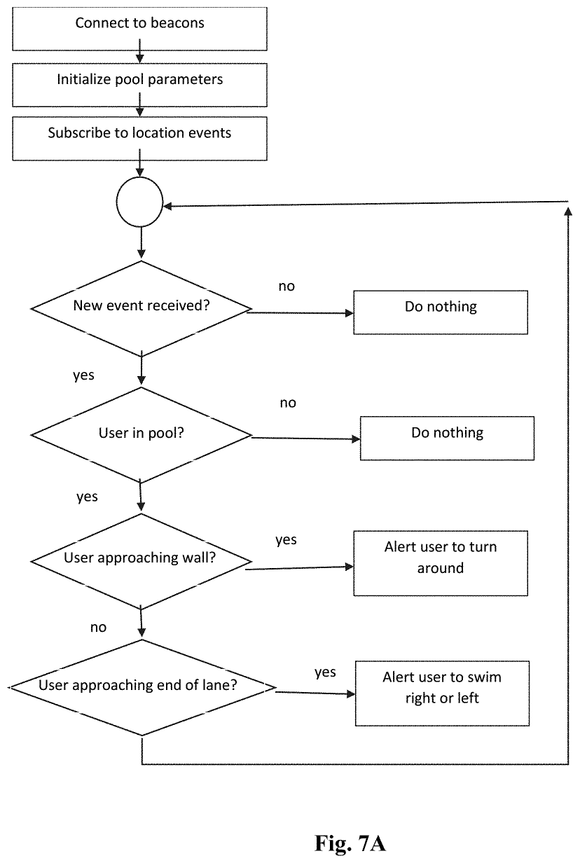

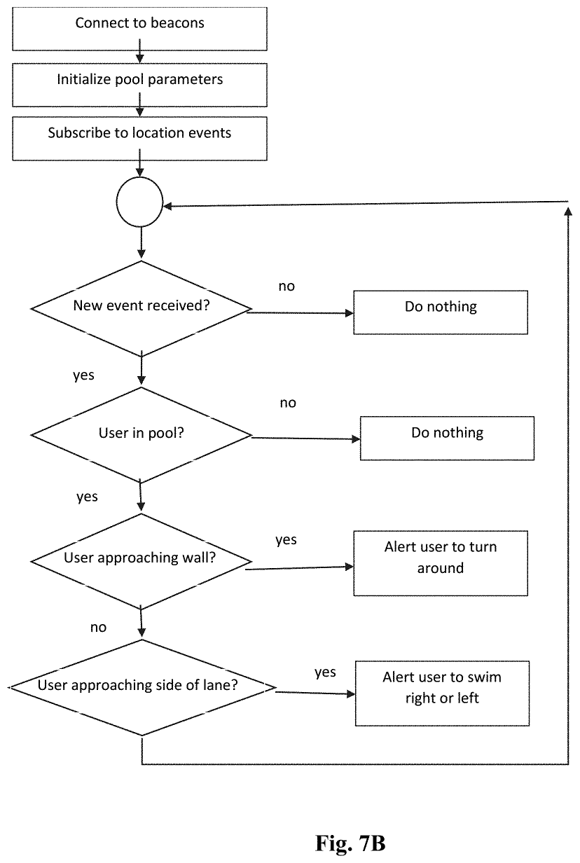

A remote processor 3 was set up in the vicinity of the pool area (i.e., Raspberry Pi 0 with a Linux operating system). A proprietary computer program (see FIGS. 7A, 7B, and 8) was written in Python to enable collection of the pool frame of reference, a swimming lane's frame of reference, the stationary beacons' signals, and the tag's signals (see FIGS. 7A, 7B, and 8). The computer program determined when a signal should be sent to the signal output device to alert the swimmer. This determination was made by comparing the swimmer's position and direction of travel and comparing them to pre-configured boundaries for the swim lane. A swimmer moving toward a first or second end of the swim lane was sent an audio alert at a fixed distance (e.g., 15 feet) from the end of the lane, indicating to the subject that it was time to change course (i.e., reverse direction). The computer program also alerted the swimmer to swim to the left or right as appropriate when it detected that the tag had strayed from the center of the lane.

### Example 1B. Ultrasonic tracking and alerting a swimmer who is swimming lengths of a pool

Stationary ultrasonic beacons (MarvelMind Super-Beacon available from Marvelmind of Tallinn, Estonia) are installed against the walls near each corner of a swimming pool. An ultrasonic tag (Beacon Mini-RX available from Marvelmind of Tallinn, Estonia) reports an x,y,z location for the tagged subject. If desired, this data can be filtered by combining it with information from a gyroscope and an accelerometer.

### Example 2. Tracking and alerting a swimmer who is swimming underwater

Four QORVO DWM1001 ultra-wideband stationary beacons 2 were installed on the walls, approximately near each corner of a swimming pool. A mobile DWM1001 UWB tag 1 is housed in a waterproof container (e.g., ZIPLOCK® bag) and inserted into a swim cap that is worn by the subject. The same swim cap also houses a wearable processor (e.g., Smartphone, Apple Watch) that combines an inertial measurement unit (IMU), a processor capable of running custom software, and is adapted to connect (e.g., wirelessly or wired) to the signal output device. The communication device is also housed in a flexible waterproof casing within the swimcap. The signal output device is a waterproof, BLUETOOTH®-enabled, Aeropex bone conduction headset. The headset is worn on or near the temples of the swimmer.

The tag and stationary beacons system reports an x,y,z location for the tagged subject, and that location is received by the Smartphone. This received data is combined with data from the communication device's own internal IMU using sensor fusion. The combined data provides a more accurate x,y,z position estimate than could be achieved using either device on its own.

Notably, the communication device's IMU could be used on its own for short distances to estimate the swimmer's position when connection with the remote processor is lost, which happens when the tag DWM1001 is underwater.

As in Example 1, the computer program calculates when a signal should be sent to the signal output device to alert the swimmer. This determination is made by using the swimmer's position and direction of travel and comparing them to pre-configured boundaries for the swim lane. A swimmer moving toward either end of the swim lane is be sent an audio alert at a fixed distance (e.g., 15 feet) from the end of the lane. The computer program could similarly alert the swimmer to swim left or right as appropriate if it determined that the swimmer had strayed from the center of the lane or has strayed from a preselected bearing.

### Example 3. A lightweight, portable method for tracking and alerting using a wearable processor in the absence of a remote processor.

An Apple Watch is described as an example of a communication device in Example 3. Notably, this watch has a built-in UWB chip. The UWB chip is capable of acting as the tag. In this scenario the Apple Watch, which is in communication with the stationary beacons, performs: the position tracking; IMU; sensor fusion; and alerting functions. The Apple Watch communicates a signal to the swimmer via a signal output device (see FIG. 6). Hence, no remote processor is needed.

### EQUIVALENTS

It will be understood by those skilled in the art that this description is made with reference to certain embodiments and that it is possible to make other embodiments employing the principles of the invention which fall within its spirit and scope.

1. A device for alerting a subject in motion of an obstacle, comprising:
a mobile transceiver (“tag”) adapted to be worn by a subject within a designated area;
a plurality of stationary transceivers (“beacons”) located in selected positions, wherein the beacons are spaced apart from each other above or around the designated area, and wherein the beacons are able to communicate with each other;
a wearable signal output device; and
a processor coupled to at least one non-transitory computer readable medium containing processing instructions that when executed by the processor cause the processor to:
receive data regarding the trilaterational position of the tag relative to at least three of the beacons;
calculate in real-time the tag's position relative to one or more selected obstacle(s) in the designated area; and
output a signal to the signal output device when the tag is approaching an undesirable location.

2. The device of claim 1, wherein the subject is a swimmer and the undesirable location comprises a location of an obstacle.

3. The device of claim 1, wherein the tag and plurality of beacons emit ultra-wideband or ultrasonic signals.

4. The device of claim 1, wherein the subject is visually-impaired.

5. The device of claim 1, wherein the signal output device is adapted to produce an audible signal.

6. The device of claim 1, wherein the signal output device is adapted to produce a haptic signal.

7. The device of claim 1, wherein the signal output device is located on or near the subject's temples or ears.

8. The device of claim 1, wherein the output device is a bone conduction headset, bone conduction BLUETOOTH® headset, or a haptic signalling device.

9. The device of claim 1, further comprising an Inertial Measurement Unit (IMU).

10. A system, comprising:
a mobile transceiver (“tag”) adapted to be attached to a subject in a designated area;
a plurality of stationary transceivers (“beacons”) that are in communication with the tag and with each other and are in spaced apart, fixed positions around the perimeter of the designated area;
a signal output device configured to be worn by the subject; and
a processor and a set of computer-readable instructions on a non-transitory computer-readable medium coupled to or associated with the processor that, when executed on the processor, cause the processor to receive data from the tag, calculate when the tag has passed a defined point and communicate with the signal output device to emit a signal.

11. The system of claim 10, wherein the subject is a swimmer and the undesirable location comprises a location of an obstacle.

12. The system of claim 10, wherein the tag and the plurality of beacons are adapted to emit and receive ultra-wideband or ultrasonic signals.

13. The system of claim 10, wherein the subject is visually-impaired.

14. The system of claim 10, wherein the output device is adapted to produce an audible signal.

15. The system of claim 10, wherein the output device is adapted to produce a haptic signal.

16. The system of claim 10, wherein the output device is located on or near the subject's temples or ears.

17. The system of claim 10, wherein the output device is a bone conduction headset, bone conduction BLUETOOTH® headset, or a haptic signalling device.

18. The system of claim 10, further comprising an Inertial Measurement Unit (IMU).

19. A method of tracking a subject in motion and alerting the subject, comprising:
disposing a mobile transceiver (“tag”) and a signal output device on a subject;
disposing a plurality of stationary transceivers (“beacons”) in spaced apart, fixed positions around the perimeter of a designated area whereby the beacons and the tag are in communication substantially consistently, whereby tag location data is provided by trilateration;
communicating the tag location data to a processor having a set of computer-readable instructions on a non-transitory computer-readable medium coupled to or associated with the processor that, when executed on the processor, direct the processor to compare the tag location data to stored parameters for an activity area; and
sending one of a plurality of output signals to a signal output device if the tag is approaching a selected location.

20. The method of claim 19, wherein the plurality of output signals comprises turn left, turn right, or reverse direction.

21. The method of claim 19 wherein the tag and the plurality of beacons emit and receive ultra-wideband or ultrasonic signals.

22. Computer executable programmed instructions stored on a non-transitory computer readable storage medium, wherein the programmed instructions direct a processor to:
receive data regarding the trilaterational position of a mobile transceiver (“tag”) relative to at least three of stationary transceivers (“beacons”) in a designated area;
calculate the tag's position relative to stored parameters for one or more selected locations in the designated area; and
output a signal to the signal output device when the tag is approaching a selected location.
